(19) United States
(12) Patent Application Publication
(10) Pub. No.: US 2004/0068321 A1
(43) Pub. Date: Apr. 8, 2004

(54) Reduced-friction artificial disc replacements

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/679,919

(22) Filed: Oct. 6, 2003

Related U.S. Application Data
(60) Provisional application No. 60/416,337, filed on Oct. 4, 2002.

(51) Int. Cl. A61F 2/44

(52) U.S. Cl. 623/17.16; 623/17.15

(57) ABSTRACT

One or more rolling or rotating elements such as rollers or bearings reduce friction in an artificial disc replacement (ADR). In the preferred embodiment, the rolling or rotating elements are situated between the ADR and the vertebrae or endplate resurfacing components. The reduced friction decreases the shear stress on the vertebral endplates which, in turn, may decrease pain from the endplates. Alternatively, when used with resurfacing components, the reduced shear will prolong the life of the ADR. Multidirectional wheels allow the ADR to move in all directions to accommodate spinal motion. Roller embodiments allow flexion and extension of the spine with movement of the ADR. Conversely, the vertebrae slide over the dome shaped rollers during lateral bending. Spinal movement occurs as the vertebrae move over the device. The mobile therefore device “self-centers.” In all embodiments, the body of the device may flex to dampen axial loads.

### REFERENCE TO RELATED APPLICATION

This application claims priority from U.S. Provisional Patent Application Serial No. 60/416,337, filed Oct. 4, 2002, the entire content of which is incorporated herein by reference.

### FIELD OF THE INVENTION

This invention relates generally to prosthetic joint components and, in particular, to reduced-friction artificial disc replacements (ADRs).

### BACKGROUND OF THE INVENTION

Many spinal conditions, including degenerative disc disease, can be treated by spinal fusion or through artificial disc replacement (ADR). Since spinal fusion eliminates motion across fused segments of the spine, the discs adjacent to the fused level are subjected to increased stress. The increased stress increases the changes of future surgery to treat the degeneration of the discs adjacent to the fusion.

ADRs offer several advantages over spinal fusion, the most important of which is the preservation of spinal motion. One of the most important features of an artificial disc replacement (ADR) is its ability to replicate the kinematics of a natural disc. ADRs that replicate the kinematics of a normal disc are less likely to transfer additional forces above and below the replaced disc. In addition, ADRs with natural kinematics are less likely to stress the facet joints and the annulus fibrosus (AF) at the level of the disc replacement. Replicating the movements of the natural disc also decreases the risk of separation of the ADR from the vertebrae above and below the ADR.

The kinematics of ADRs are governed by the range of motion (ROM), the location of the center of rotation (COR) and the presence (or absence) of a variable center of rotation (VCOR). Generally ROM is limited by the facet joints and the AF. Motion across prior-art prosthetic joints occurs by rotation and sliding between the components. The resultant friction causes surface wear leading to problems well known to orthopedic surgeons (i.e., fracture of polyethylene trays, polyethylene debris, component loosening, etc.). “Revision” surgery is frequently required to correct the problems associated with component wear.

### SUMMARY OF THE INVENTION

Broadly, this invention uses one or more rolling or rotating elements such as rollers or bearings to reduce the friction in an artificial disc replacement (ADR). In the preferred embodiment, the rolling or rotating elements are situated between the ADR and the vertebrae or endplate resurfacing components. The reduced friction decreases the shear stress on the vertebral endplates which, in turn, may decrease pain from the endplates. Alternatively, when used with resurfacing components, the reduced shear will prolong the life of the ADR.

In many embodiments, the inventive ADR will be tethered using a “mobile link” of the type described in co-pending U.S. patent application Ser. No. 10/426,995, the entire content of which is incorporated herein by reference. Other embodiments of the device use multidirectional, caster-like wheels not unlike those found on office chairs. The multidirectional wheels allow the ADR to move in all directions to accommodate spinal motion. The roller embodiments allow flexion and extension of the spine with movement of the ADR. Conversely, the vertebrae slide over the dome shaped rollers during lateral bending.

A preferred embodiment utilizes a spacer with wheels that rotate in all directions. Wheel rotation occurs about a transverse axle and a vertical axle that connects the wheel and transverse axle to the body of the device. Spinal movement occurs as the vertebrae move over the device. The mobile therefore device “self-centers.” The body of the device could flex slightly to dampen axial loads.

The advantages of the invention are many. The use of rotating elements allows the device to move quickly to accommodate/allow spinal flexion and extension while reducing friction between the device and the vertebral endplates or between the device and the resurfacing components. Reduced friction on the vertebral endplates could reduce pain from the endplates. Reduced friction on the resurfacing components would increase the lifespan of the device. The use of dome-shaped rollers allow at least 5 degrees of lateral bending in either direction.

In all embodiments, a seal could be used to trap debris inside the ADR. The seal could surround the periphery of the superior ADR EP and the inferior ADR EP. The seal could also hold a fluid within the ADR. Various fluids or lubricants may be used, including but not limited to: water or aqueous solutions, triglyceride oil, soybean oil, an inorganic oil (e.g. silicone oil or fluorocarbon), glycerin, ethylene glycol, or other animal, vegetable, synthetic oil, or combinations thereof could be used. The seal could be made of an expandable elastomer such as those used in medical devices for the cardiovascular system.

### BRIEF DESCRIPTION OF THE DRAWINGS

FIG. 1 is a view of the lateral side of a device constructed in accordance with this invention;

FIG. 2 is a view of the top of the device shown in FIG. 1;

FIG. 3 is a view of the bottom of the device of FIG. 1;

FIG. 4 is a view of the lateral aspect of the spine and the device;

FIG. 5 is a view of the anterior aspect of the spine and the device;

FIG. 6 is a view of the anterior aspect of the spine and an ADR that uses rollers which rotate around axles;

FIG. 7 is view of the lateral portion of the spine and the ADR;

FIG. 8 is a view of the lateral aspect of the device with the lateral connecting piece on the device of the present invention;

FIG. 9 is a view of the lateral aspect of the device with the lateral plate;

FIG. 10 is an anterior view of the spine and the device between the optional endplate;

FIG. 11 is a lateral view of the spine and the device with the optional endplate resurfacing components.

FIG. 12A is a view of the side an alternative component with bearings;

FIG. 12B is a coronal cross-section of the embodiment of the device shown in FIG. 12A;

FIG. 12C is a view of the top of the embodiment of the device shown in FIG. 12A;

FIG. 12D is a coronal cross-section of an alternative embodiment of the mobile component shown in FIG. 12B; and

FIG. 12E is a coronal cross-section of an alternative embodiment of the device drawn in FIG. 12D.

### DETAILED DESCRIPTION OF THE INVENTION

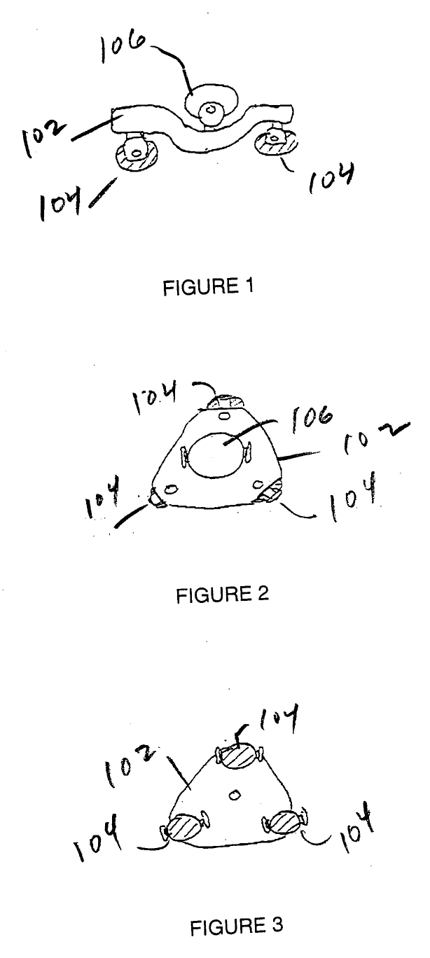

FIG. 1 is a view of the lateral side of a device constructed in accordance with this invention. The device has a body 102, three wheels 104 on the inferior surface and a wheel 106 on the superior surface. FIG. 2 is a view of the top of the device, and FIG. 3 is a view of the bottom of the device. Alternative embodiments with a different number of inferior/superior wheels are possible.

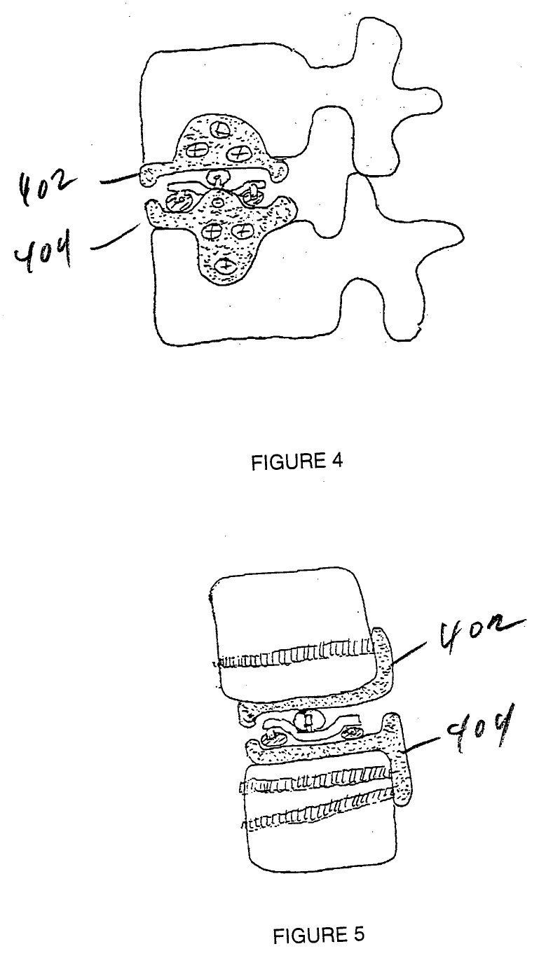

FIG. 4 is a view of the lateral aspect of the spine and the device. The endplate resurfacing components 402, 404 have features, raised edges, to contain the mobile portion of the device. FIG. 5 is a view of the anterior aspect of the spine and the device. The mobile portion of the device could also be connected to the inferior resurfacing component via a cable, as described in my co-pending U.S. provisional patent application Serial No. 60/376,505, the entire content of which is incorporated herein by reference.

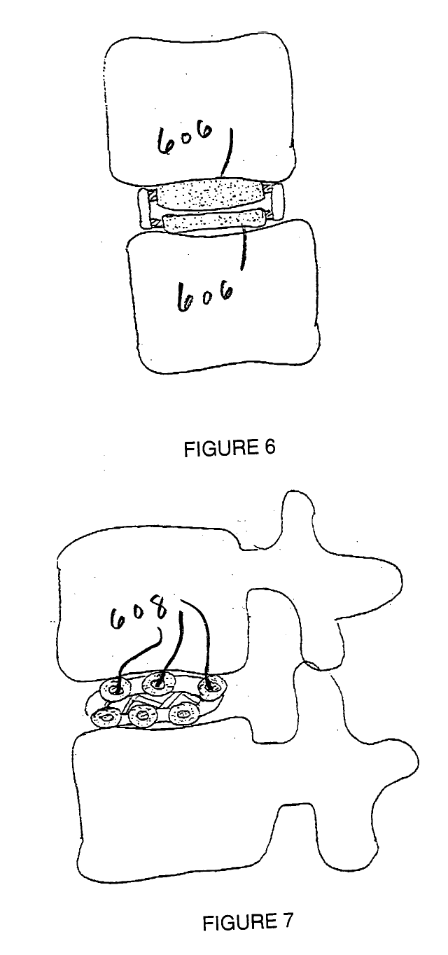

FIG. 6 is a view of the anterior aspect of the spine and an ADR that uses rollers 606 which rotate around axles 608. Alternatively, the rollers can have axle shaped projections from the ends that rotate in the lateral connecting pieces. FIG. 7 is view of the lateral portion of the spine and the ADR. The near lateral connection piece has been removed to show the inside of the roller portion of the ADR.

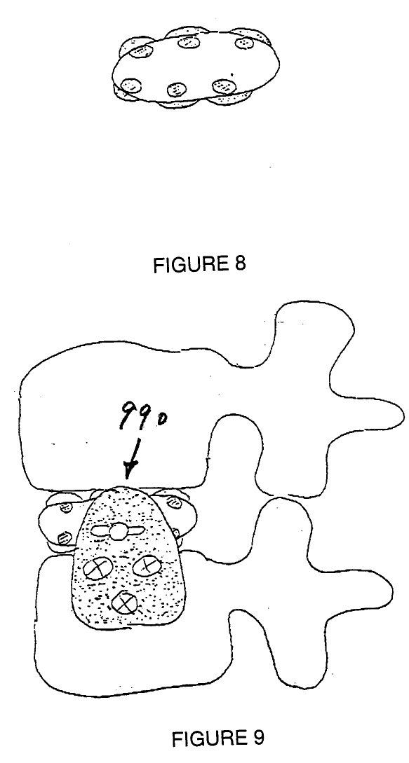

FIG. 8 is a view of the lateral aspect of the device with the lateral connecting piece on the device. FIG. 9 is a view of the lateral aspect of the device with the lateral plate 990 and a mobile link of the type referenced in the Summary of the Invention. The drawing also illustrates the use of a slot in the lateral plate. The slot allows the mobile link to travel anterior to posterior, thus increasing the mobility of the ADR in an anterior to posterior direction, without increasing the mobility of the device in a left to right direction.

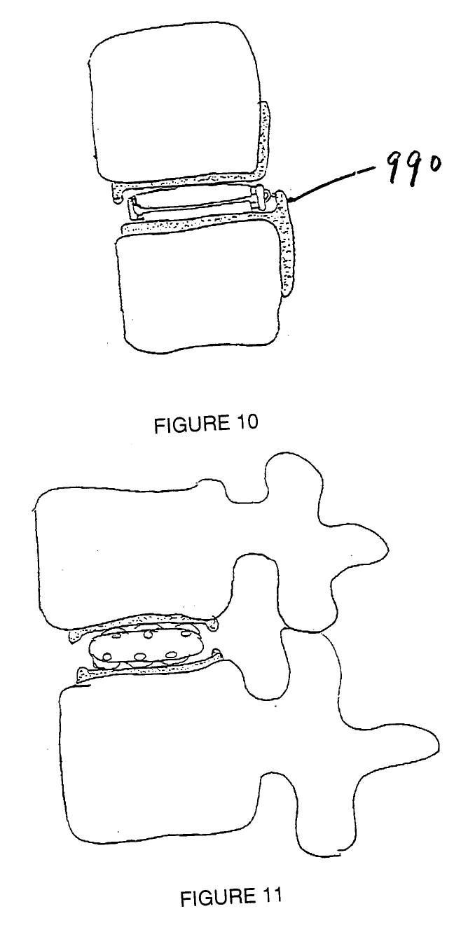

FIG. 10 is an anterior view of the spine and the device between the optional endplate resurfacing components described in previous disclosures. The endplate resurfacing components may preferably include raised edges to prevent the ADR from extruding or placing pressure on the Annulus Fibrosis (AF). Pressure on the AF could lead to tearing of the AF and pain.

FIG. 11 is a lateral view of the spine and the device with the optional endplate resurfacing components. The lateral plate portion of the interior resurfacing component was not drawn to better illustrate the raised anterior and posterior edges of the resurfacing components. As discussed previously, the lateral plate could extend from the superior endplate component to facilitate insertion at the L4/L5 level (the screws go into the body of L4 rather than L5). Also as previously described, the plate fits over the anterior portion of S1 at the L5/S1 level and the anterior aspect of the cervical spine.

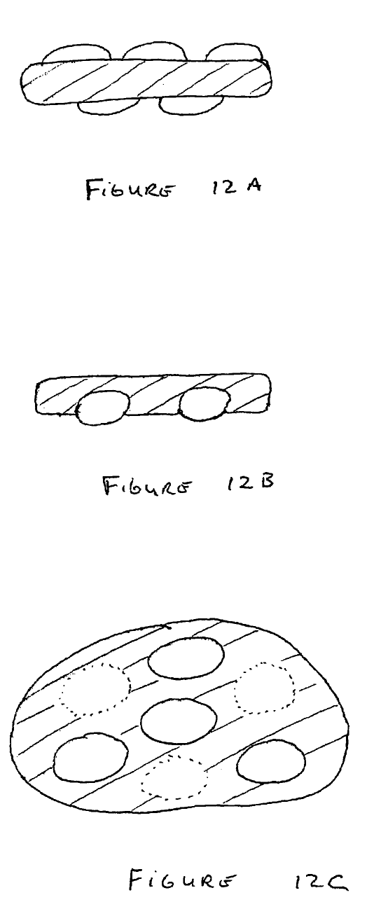

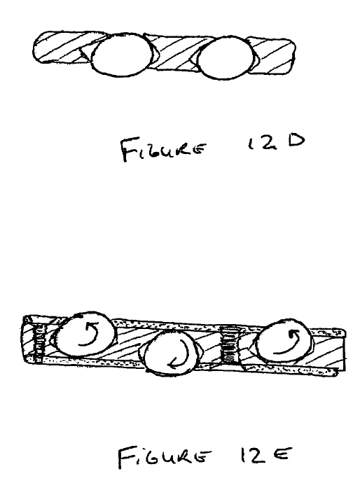

FIG. 12A is a view of the side an alternative component with bearings. Unlike the component drawn in FIG. 1, this embodiment of the device does not utilize axles; instead, the bearings are housed in a component with spherical holes. The bearings articulate with ADR EPs or the vertebral endplates and the component that contains the bearings. Some of the bearings articulate with the superior ADR EP or superior vertebra. Other bearings articulate with the inferior ADR EP or inferior vertebra. A single bearing articulates with either the superior or the inferior portion of the device but not both the superior and inferior portions of the device.

FIG. 12B is a coronal cross-section of the embodiment of the device drawn in FIG. 12A. The device was cross-sectioned through some bearings that articulate with inferior portion of the device. FIG. 12C is a view of the top of the embodiment of the device drawn in FIG. 12A. The bearings that articulate with the superior portion of the device are represented by the circles with solid lines. The bearings that articulate with the inferior portion of the device are represented by the circles with dotted lines.

FIG. 12D is a coronal cross-section of an alternative embodiment of the mobile component drawn in FIG. 12B. The device was cross-sectioned through some of the bearings that articulate with the inferior portion of the device. The inferior bearings protrude through the inferior portion of the device more than they protrude through the superior portion of the device. The superior bearings protrude through the superior portion of the device more than they protrude through the inferior portion of the device. The bearings articulate with either the superior ADR EP or the inferior ADR EP.

FIG. 12E is a coronal cross-section of an alternative embodiment of the device drawn in FIG. 12D. The bearings are housed in three separate components. The components that house the bearings are connected, for example by screws. Use of more than one housing component assists with the assembly of the device. The device of FIG. 12 allows the bearings on the superior surface of the device to rotate in a different direction than the bearings on the inferior surface of the device.

In all embodiments, a seal could be used to trap debris inside the ADR. The seal could surround the periphery of the superior ADR EP and the inferior ADR EP. The seal could also hold a fluid within the ADR. Various fluids or lubricants may be used, including but not limited to: water or aqueous solutions, triglyceride oil, soybean oil, an inorganic oil (e.g. silicone oil or fluorocarbon), glycerin, ethylene glycol, or other animal, vegetable, synthetic oil, or combinations thereof could be used. The seal could be made of an expandable elastomer such as those used in medical devices for the cardiovascular system.

I claim:

1. A reduced-friction artificial joint replacement (ADR), comprising:
a first endplate component adapted for fixation to an upper vertebral body;
a second endplate component adapted for fixation to an upper vertebral body; and
a wheel, bearing or other rotating element between the two endplate components to reduce friction during spinal flexion, extension or other movements.

2. The reduced-friction artificial joint replacement (ADR) of claim 1, including one or more rotating elements disposed on casters.

3. The reduced-friction artificial joint replacement (ADR) of claim 1, including one or more elements rotatable about axes oriented generally medial to lateral.
